(19) United States
(12) Patent Application Publication
(10) Pub. No.: US 2024/0294943 A1
(43) Pub. Date: Sep. 5, 2024

(54) METHOD FOR IMPROVING GENETIC ENGINEERING OF ADOPTIVE CELL THERAPIES

(71) Applicant: CHARLES RIVER LABORATORIES, INC., Wilmington, MA (US)

(72) Inventors: Despina PLEITEZ, Wilmington, MA (US); Terri JERBI, Wilmington, MA (US); Jade SCHEERS, Wilmington, MA (US); Sarah CAMPION, Wilmington, MA (US); Matthew HEWITT, Wilmington, MA (US); Alex SARGENT, Wilmington, MA (US)

(21) Appl. No.: 18/593,114

(22) Filed: Mar. 1, 2024

Related U.S. Application Data
(60) Provisional application No. 63/488,354, filed on Mar. 3, 2023.

(51) Int. Cl. C12N 15/87; C12N 15/113

(52) U.S. Cl. CPC C12N 15/87; C12N 15/113; C12N 2310/11; C12N 2310/14; C12N 2310/141

(57) ABSTRACT

The present disclosure provides methods for electroporating cells, in particular cells useful in cellular-based therapies, that increase cell viability, cell proliferation and transfection efficiency. The methods suitably utilize a post-electroporation supplement to improve the genetic engineering of the various cells.

### FIELD OF THE INVENTION

The present disclosure provides methods for electroporating cells, in particular cells useful in cellular-based therapies, that increase cell viability, cell proliferation and transfection efficiency. The methods suitably utilize a post-electroporation supplement to improve the genetic engineering of the various cells.

### BACKGROUND OF THE INVENTION

Genetic engineering is an essential but challenging part of creating safe and effective cell therapies. A popular approach to genetically modify cells is to use electroporation (EP) to create transient pores in the cell membrane and introduce mRNA, DNA, endonucleases, and/or transposon systems into the cell. While electroporation can result in high gene editing and transfection efficiency, it often results in high cell death and a significant drop in cell viability.

What is needed are methods that improve cell recovery and viability, as well as transfection efficiency, to increase the adoption and success of cellular-based therapies. The present invention fulfils these needs.

### SUMMARY OF THE INVENTION

In embodiments, provided herein is a method of electroporating a cell, comprising suspending the cell in an electroporation buffer to create a cell suspension, subjecting the cell to one or more electroporation pulses, and adding a post-electroporation supplement to the cell suspension, wherein the post-electroporation supplement comprises a lipid enriched, human serum.

In further embodiments, provided herein is a method of transfecting a genetic construct into a cell, comprising suspending the cell in an electroporation buffer to create a cell suspension, introducing the genetic construct into the cell suspension, subjecting the cell to one or more electroporation pulses, and adding a post-electroporation supplement to the cell suspension, wherein the post-electroporation supplement comprises a lipid enriched, human serum.

Also provided herein is a composition comprising a lipid enriched, human AB serum, wherein the composition increases cell viability of an electroporated cell population.

### BRIEF DESCRIPTION OF THE DRAWINGS

FIG. 1 shows cell viability 24 hours post electroporation.

FIG. 2 shows cell number 24 hours post electroporation.

FIG. 3 shows transgene expression 4 days post electroporation.

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps.

Electroporation is a widely-used method for permeabilization of cell membranes by temporary generation of membrane pores with electrical stimulation. The applications of electroporation include the delivery of various genetic constructs, including DNA, RNA and siRNA, as well as peptides, proteins, antibodies, drugs or other substances into a variety of cells.

During electroporation, cells are suspended in a buffer or medium. The cell suspension is then placed in a cuvette, chamber, cartridge, or other device, and an electric discharge is applied to the cell suspension, and thus the cells within the suspension, to induce the permeabilization effect. Various parameters for carrying out electroporation, including the strength of the electric discharge, the length and number of pulses of discharge, waiting periods between discharges, etc., are known in the art.

The ability to effectively deliver a target substance into a cell, while still maintaining the viability and proliferability of the cell, is a challenge in the field of electroporation. The electroporation process is usually toxic to the cells. First, when the electric field strength is too high, the cell membranes may be irreversibly damaged. Secondly, while electrically induced membrane pores allow a target substance to enter the cells, the pores may also allow outflow of cellular contents and inflow of other unintended substances which could negatively affect cell viability. Thirdly, the heat generated by the electric current may harm the cells. Lastly, electrochemically generated toxic agents such as free radicals, gas and metal ions near the electrodes can be harmful to the cells.

As described herein, it has been determined that the use of a post-electroporation supplement can significantly increase cell recovery and viability, while still maintaining, or even increasing, the effectiveness of target substance delivery.

In embodiments, provided herein is method of electroporating a cell, comprising suspending the cell in an electroporation buffer to create a cell suspension; subjecting the cell to one or more electroporation pulses; and adding a post-electroporation supplement to the cell suspension, wherein the post-electroporation supplement comprises a lipid enriched, human serum.

As used herein “electroporating” or “electroporation” refers to the action or process of introducing a target substance into a cell using one or more pulses of electricity to briefly open pores in the cell membrane. As described herein, a cell, suitably a population of cells all of the same type of cell and/or from the same source, are suspended in an electroporation buffer. Various electroporation buffers are known in the art and suitably include a buffer system as a base (e.g., HEPES (N-2-hydroxyethylpiperazine-N′-2-ethanesulfonic acid), sodium phosphate, phosphate buffered saline (PBS), etc.) as well as sugars (e.g., sucrose, trehalose), and various salts, such as MgCl₂, KCl, MgSO₄, or a MgCl₂/KCl mixture. Other additives can also be included.

The cell suspension, and thus the cells themselves, are subjected to one or more electroporation pulses. Pulse strength, duration, timing, as well as pauses between pulses, can be determined by those of skill in the art and are generally tailored to the type of cell being electroporated, the buffers being used, the target substance being introduced, as well as other factors. Generally, smaller sized cells (e.g., between 10-25 microns in diameter) require higher applied voltages (e.g. between 500-1200 volts, depending on the temperature of the environment/aqueous solution) to achieve successful membrane permeation, while larger sized cells (e.g., between 25-50 microns in diameter) require lower applied voltages (e.g. between 100 to 500 volts, depending on the temperature of the environment/aqueous solution) for successful membrane permeation. Pulse durations generally last between 5 to 800 microseconds.

As described herein, following the electroporation, a post-electroporation supplement is added to the cell suspension. In embodiments, this post-electroporation supplement comprises a lipid enriched, human serum.

Suitably, the post-electroporation supplement is added within about 30 minutes following the end of the electroporation protocol (i.e., before 30 minutes has elapsed following the end of the electroporation protocol). More suitably, the post-electroporation supplement is added within about 25 minutes following the end of the electroporation protocol, or within about 20 amounts, within about 15 minutes, without about 10 minutes, within about 5 minutes, or between about 5 minutes to about 30 minutes, between about 5 minutes to about 20 minutes, or between about 5 minutes to about 10 minutes. In other embodiments, the post-electroporation supplement is added after about 30 minutes following the end of the electroporation protocol, but before about 1 hour following the end of the protocol.

The methods described herein can be utilized with any cell, including a mammalian cell (including human cells) as well as insect cells. As used herein, a cell and a cell population, which includes a plurality of cells, are used interchangeably. In suitable embodiments, the cell is a cell useful in a therapeutic application in a mammal, such as a human cell-based therapy. Exemplary cells include T-cells, hematopoietic stem cells (HSCs), mesenchymal stem cells (MSCs), natural killer cells (NK cells), etc. Suitably, the source of the human cell is a patient that is also being treated with the therapy (an autologous therapy). In other embodiments, the cell can be from a donor and can be used for one or more other patients (an allogeneic therapy).

Various methods can be used to produce the post-electroporation supplement. For example, the lipid enriched, human serum can be prepared by heat inactivating human serum to thereby enrich the serum with lipids. For example, the human serum can be heated to a temperature of about 50° C. to about 80° C. for about 10 minutes to about 50 minutes. Following the heating, the human serum can be centrifuged through a filter so as to collect the lipid enriched human serum. Suitably, the human serum is heated to a temperature of about 60° C. to about 80° C. for about 20 minutes to about 40 minutes, more suitably the human serum is heated to a temperature of about 70° C. for about 30 minutes. In exemplary embodiments the heat inactivated human serum is passed through a 0.2 μm filter to yield the heat inactivated, lipid enriched human serum.

In other embodiments, the human serum is passed through a column to concentrate the lipids within the serum and the lipid enriched human serum is collected. For example, the human serum is passed through a silica column. In embodiments, a column is prepared from fumed silica powder, and human serum is added to the column. The human serum is then centrifuged through the column, and delipidated serum (the aqueous top layer) is removed to yield a lipid enriched human serum layer.

In exemplary embodiments, the human serum that is utilized in the preparation of the lipid enrich human serum is human AB serum, i.e., human serum from an AB positive human donor that lacks antibodies specific for both A and B blood-type antigens, making it suitable for transplantation and cell therapy. Other human serum can also be used.

In suitable embodiments, the post-electroporation supplement is added following electroporation of the cells at about 1% to about 20% by volume of the cell suspension. More suitably, the post-electroporation supplement is added at about 2% to about 15%, or about 2% to about 10%, about 3% to about 7%, about 4% to about 6%, or about 4%, about 5%, about 6%, about 7%, about 8%, about 9% or about 10% by volume of the cell suspension.

As described herein, it has been surprisingly found that addition of a post-electroporation supplement increases cell recovery and leads to greater viability of cells, following electroporation.

For example, in embodiments, the viability of a cell (or a cell population) receiving a post-electroporation supplement is at least about 10% greater than a cell that has not received the post-electroporation supplement, following electroporation. In embodiments, the viability of a cell receiving the post-electroporation is at least about 20% greater, at least about 30% greater, at least about 40% greater, or about 5% to about 40% greater, about 10% to about 30% greater, about 20% to about 40% greater, about 20% to about 30% greater, about 30% to about 40% greater, or about 20% greater, about 30% greater, or about 40% greater, than a cell that has not received the post-electroporation supplement, following electroporation.

The methods described herein have also been surprisingly found to increase the number of viable cells recovered following electroporation, in comparison to cells that have not received the post-electroporation supplement, following electroporation, and are nearly comparable to the viable number of cells recovered without any electroporation. In embodiments, a viable number of cells is at least about 1.1-fold greater than a cell population that has not received the post-electroporation supplement, following electroporation, or at least about 1.2-fold greater, at least about 1.3-fold greater, at least about 1.4-fold greater, at least about 1.5-fold greater, at least about 1.6-fold greater, at least about 1.7-fold greater, at least about 1.8-fold greater, at least about 1.9-fold greater, at least about 2-fold greater, or about 1.1-fold to about 3-fold greater, about 1.2-fold to about 2-fold greater, about 1.3-fold to about 1.8-fold greater, about 1.4-fold to about 1.6-fold greater, or about 1.2-fold greater, about 1.3-fold greater, about 1.4-fold greater, about 1.5-fold greater, about 1.6-fold greater, about 1.7-fold greater, about 1.8-fold greater, about 1.9-fold greater, or about 2-fold greater, in comparison to cells that have not received the post-electroporation supplement following electroporation.

In further embodiments, provided herein is a method of transfecting a genetic construct into a cell, comprising, suspending the cell in an electroporation buffer to create a cell suspension, introducing the genetic construct into the cell suspension, subjecting the cell to one or more electroporation pulses, and adding a post-electroporation supplement to the cell suspension, wherein the post-electroporation supplement comprises a lipid enriched, human serum.

In addition to increases in cell recovery and viability, it has also been surprisingly found that the transfection efficiency of cells receiving the post-electroporation supplement is greater than cells that do not receive the post-electroporation supplement, following electroporation.

As used herein, the term “transfecting” refers to introducing a genetic construct into a cell, but does not require the use of a viral vector or plasmid to enter the cell. A “genetic construct” refers to an artificially designed or engineering nucleic acid. Examples of genetic constructs that can be transfected into the cells include, but are not limited to, RNA, messenger RNA (mRNA), antisense RNA, small inhibitory RNA (siRNA), microRNA, DNA, as well as DNA contained within a viral vector or a viral plasmid. In embodiments, the genetic construct can also include a transposon (transposable element), including DNA transposons, retrotransposons, autonomous or nonautonomous transposons, etc. In additional embodiments, the genetic construct can comprise an endonuclease, i.e., an enzyme that cleaves a polynucleotide chain, including Type I, II and III endonucleases.

The methods described herein can also be used to deliver other target substances, such as proteins, peptides, amino acids, endosomes, etc., into the cells.

As described herein, the viability of the cells that receive the post-electroporation supplement is increased, suitably the viability of the cell is at least 30% greater than a cell that has not received the post-electroporation supplement. In other embodiments, a viable number of cells is at least 1.5-fold greater than a cell that has not received the post-electroporation supplement.

In embodiments where the cells are transfected with a genetic construct, it has been surprisingly found that a transfection efficiency of the cells is at least 5% greater than a cell that has not received the post-electroporation supplement, following the electroporation. Transfection efficiency as used herein refers to the percentage of cells within a population that express or contain a delivered target substance, suitably a protein expressed by a genetic construct, following an electroporation procedure. For example, the transfection efficiency is at least 7% greater, at least 8% greater, at least 9% greater, at least 10% greater, at least 11% greater, at least 12% greater, at least 13% greater, at least 14% greater, at least 15% greater, at least 16% greater, at least 17% greater, at least 18% greater, at least 19% greater, at least 20% greater, or about 5% to about 25% greater, about 5% to about 20% greater, about 5% to about 15% greater, about 10% to about 15% greater, or about 5% greater, about 6% greater, about 7% greater, about 8% greater, about 9% greater, about 10% greater, about 11% greater, about 12% greater, about 13% greater, about 14% greater, about 15% greater, about 16% greater, about 17% greater, about 18% greater, about 19% greater, or about 20% greater, than a cell that has not received the post-electroporation supplement, following the electroporation.

Examples of cells that can be transfected using the disclosed methods are described herein, and include for example, a T-cells and hematopoietic stem cells, as well as MSCs, NK cells, etc.

Exemplary post-electroporation supplements for use in the transfection methods include lipid enriched, human AB serum, as described herein. Methods of preparing such lipid enriched, human AB serum are also described herein. Suitably, the post-electroporation supplement is added at about 2% to about 10% by volume of the cell suspension, suitably about 3% to 7% by volume.

Also provided herein is a composition comprising heat inactivated, lipid enriched, human AB serum, wherein the composition increases cell viability of an electroporated cell population. Methods of preparing the heat inactivated, lipid enriched human AB serum include heating human AB serum to a temperature of about 60° C. to about 80° C. for about 20-45 minutes. Other methods include passing human AB serum through a silica column and recovering the lipid enriched, human AB serum. These methods are described herein and in the Examples.

### Embodiments

In embodiments, provided herein in Embodiment 1 is a method of electroporating a cell, comprising suspending the cell in an electroporation buffer to create a cell suspension, subjecting the cell to one or more electroporation pulses; and adding a post-electroporation supplement to the cell suspension, wherein the post-electroporation supplement comprises a lipid enriched, human serum.

Embodiment 2 includes the method of Embodiment 1, wherein viability of the cell is at least 30% greater than a cell that has not received the post-electroporation supplement.

Embodiment 3 includes the method of Embodiment 1 or Embodiment 2, wherein a viable number of cells is at least 1.5-fold greater than a cell that has not received the post-electroporation supplement.

Embodiment 4 includes the method of any of Embodiments 1 to 3, wherein the cell is a T-cell.

Embodiment 5 includes the method of any of Embodiments 1 to 3, wherein the cell is a hematopoietic stem cell.

Embodiment 6 includes the method of any of Embodiments 1 to 5, wherein the post-electroporation supplement comprises lipid enriched, human AB serum.

Embodiment 7 includes the method of any of Embodiments 1 to 6, wherein the post-electroporation supplement is added at about 2% to about 10% by volume of the cell suspension.

Embodiment 8 is a method of transfecting a genetic construct into a cell, comprising suspending the cell in an electroporation buffer to create a cell suspension; introducing the genetic construct into the cell suspension; subjecting the cell to one or more electroporation pulses; and adding a post-electroporation supplement to the cell suspension, wherein the post-electroporation supplement comprises a lipid enriched, human serum.

Embodiment 9 includes the method of Embodiment 8, wherein viability of the cell is at least 30% greater than a cell that has not received the post-electroporation supplement.

Embodiment 10 includes the method of Embodiment 8 or Embodiment 9, wherein a viable number of cells is at least 1.5-fold greater than a cell that has not received the post-electroporation supplement.

Embodiment 11 incudes the method of any of Embodiments 8 to 10, wherein a transfection efficiency of the cell is at least 15% greater than a cell that has not received the post-electroporation supplement.

Embodiment 12 incudes the method of any of Embodiments 8 to 11, wherein the cell is a T-cell.

Embodiment 13 incudes the method of any of Embodiments 8 to 11, wherein the cell is a hematopoietic stem cell.

Embodiment 14 incudes the method of any of Embodiments 8 to 13, wherein the post-electroporation supplement comprises a lipid enriched, human AB serum.

Embodiment 15 incudes the method of any of Embodiments 8 to 14, wherein the genetic construct is RNA or mRNA.

Embodiment 16 incudes the method of Embodiment 15, wherein the RNA is antisense RNA, siRNA or microRNA.

Embodiment 17 incudes the method of any of Embodiments 8 to 14, wherein the genetic construct is DNA.

Embodiment 18 incudes the method of any of Embodiments 8 to 14, wherein the genetic construct is a viral vector or a viral plasmid.

Embodiment 19 incudes the method of any of Embodiments 8 to 14, wherein the genetic construct comprises a transposon.

Embodiment 20 incudes the method of any of Embodiments 8 to 14, wherein the genetic construct comprises an endonuclease.

Embodiment 21 incudes the method of any of Embodiments 8 to 20, wherein the post-electroporation supplement is added at about 2% to about 10% by volume of the cell suspension.

Embodiment 22 is a composition comprising a lipid enriched, human AB serum, wherein the composition increases cell viability of an electroporated cell population.

Embodiment 23 includes the composition of Embodiment 22, prepared by heating human AB serum to a temperature of about 60° C. to about 80° C. for about 20-45 minutes.

Embodiment 24 includes the composition of Embodiment 22, prepared by passing human AB serum through a silica column and recovering the lipid enriched, human AB serum.

### EXAMPLES

### Example 1: Preparation of Lipid Enriched Human Serum

Two methods have been developed for the preparation of the lipid enriched human serum for use as the post-electroporation supplement as described herein.

### Materials

The following human AB serums were utilized in the following methods:
Human AB serum (SIGMA ALDRICH®), Catalog #H4522-100 mL
cGMP Human AB serum (AKRON®), Catalog #AR1010-0100

Additional materials include:
Silica, fumed power (SIGMA ALDRICH®), Catalog #S5130-25G

### Protocols

### Heat Inactivation of Human AB Serum to Produce Lipid Enriched Human AB Serum

Lipid enriched human AB serum is prepared by heat inactivation. Human AB serum was incubated at 70° Celsius for 30 minutes in a water bath, followed by centrifugation at 500×g for 10 minutes through a 0.2 uM sterile filter. The resulting serum is lipid enriched and can be used immediately or stored at 4° C.

### Silica Extraction of Human AB Serum to Produce Lipid Enriched Human AB Serum

0.5 g fumed silica power was added to a 50 mL conical tube. 10 mL human AB serum was then added to the same tube. The tube was gently inverted and rotated intermittently (every 15-30 minutes) for 2 hours at room temperature.

The tube was then centrifuged at 4000 rpm for 10 minutes.

Delipidated plasma (top aqueous layer) was then removed with gentle aspiration.

Lipid enriched human AB serum forms as a layer at the bottom of the tube. The resulting serum can be used immediately or stored at 4° Celsius for up to one week prior to use.

### Example 2: Electroporation and Post-Electroporation Supplement Addition

Two Cryopreserved human Pan T-cell populations were sourced from Charles River Laboratories Cell Supply (Catalog #PB03C-3). Cryopreserved hematopoietic stem cells (HSCs) were obtained from internal Charles River Laboratories supplies (isolated via CliniMACS® Prodigy; >90% CD34+).

### Human T-Cell Media Comprised:

LONZA® XVivo 15 Media (BE02-060Q)
100 IU recombinant human IL-2 (MILTENYI®, 130-097-743)

### Human HSC Media Comprised:

CELLGENIX® GMP SCGM Media (20802-0500)

Cells were thawed, diluted in respective media (10-25 mL), and centrifuged at 350×g for 5 minutes. Cells were then washed twice in respective media prior to seeding into 6 well plates (FALCON®) at a respective density of: 1e⁶ cells/mL T-cells; 0.5e⁶ cells/mL HSCs.

One day after thawing, cells were centrifuged and resuspended in 100 μl of supplemented P3 Buffer per the manufacturer's protocol for Nucleofection (LONZA® P3 Nucleofection kit, V4XP-3024). 10 ng of pMax green fluorescent protein (GFP) expressing plasmid was added to each reaction tube.

5-10e⁶ T-cells or HCSs in 100+/−10 μl of P3 buffer (+GFP) were each loaded into the Lonza Nucleofector cuvettes. Cells were electroporated using the recommended protocol on the Lonza Nucleofector (both high frequency voltage pulse stimulation):
T-cells: Unstimulated T-cell program (EO-115)
HSCs: human HSC program (X)

For each cell type, 5e⁶ cells were loaded into a cuvette that was not electroporated for an untransfected control.

Immediately after nucleofection cells were collected into a 15 mL conical tube and 10 mL of each respective media was added to the tube.

Cells were then reseeded into 6-well plates (FALCON®) at a respective density of 1e⁶ cells/mL for T-cells and 0.5e⁶ cells/mL for HSCs, both with a volume of 2 mL per well.

The following supplements were then added to respective wells of T-cells and HSCs at the indicated concentrations:
Lipid enriched Human AB serum, 100 ul (5%)
No supplement (control)

24 hours post post-electroporation, cells were sampled for cell density and viability (NC200). 4 days post-electroporation, cells were sampled and run on a BD FACS Lyric flow cytometer to check for GFP expression.

Cell viability, the number of cells, and transfection efficiency were then calculated.

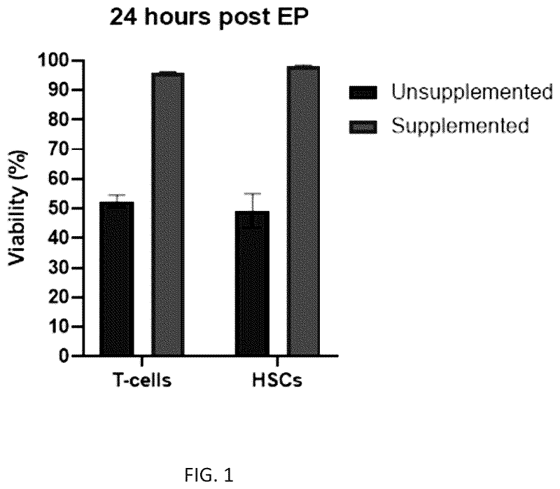

As shown in FIG. 1, 24 hours post electroporation, the viability of the T-cell population was more than 30-40% greater for the post-electroporation supplement group than the unsupplemented cells. HCSs also showed an increase of more than 30-40% viability for the supplemented versus unsupplemented cells.

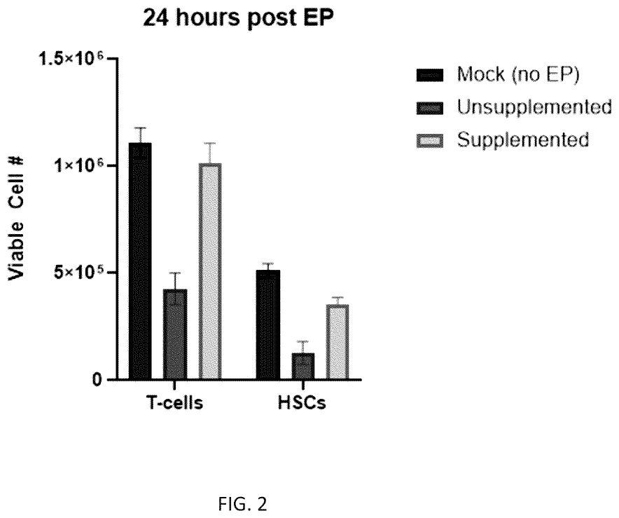

FIG. 2 shows the viable cell number for mock (no electroporation), as well as supplemented (received post-electroporation supplement) and unsupplemented cell groups. As shown, 24 hours after electroporation, the viable numbers of cells for the supplemented groups of both T-cells and HSCs were more than 1.5-fold higher than the unsupplemented cells, and reached nearly the levels of the non-electroporated cells. Thus, cell recovery increased from 50% to 80%-100% when the cells were supplemented. The pro-survival effects of the supplement are not cell type specific, as similar improvements in cell viability and recovery were observed for both T-cells and HSCs.

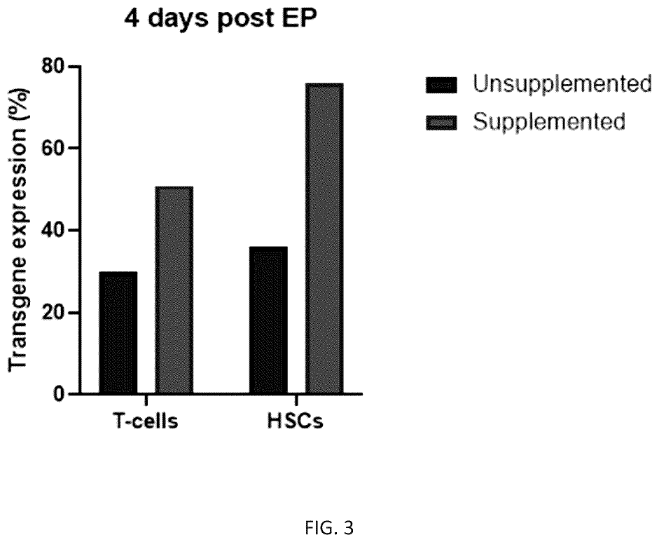

Finally, FIG. 3 shows the transfection efficiency for the T-cells and HSCs, 4 days after the transfection via electroporation. As noted, GFP expression (transgene expression) was more than 15% higher for both cell types when comparing supplemented to unsupplemented cells, approaching over 20%+ for the HSC supplemented population. Higher cell recovery in supplemented media was also associated with up to 2-fold higher transgene expression in both T-cells and HSCs, as transfected cells are better able to survive and expand.

Collectively, these results demonstrate the significant and unexpected improvements that supplementation of cells with the post-electroporation supplement described herein can have on cell recovery, viability, and transfection efficiency after electroporation. This provides a significant mechanism to improve genetic engineering of adoptive cell therapies for the treatment of cancer and other diseases.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

What is claimed is:

1. A method of electroporating a cell, comprising:
a. suspending the cell in an electroporation buffer to create a cell suspension;
b. subjecting the cell to one or more electroporation pulses; and
c. adding a post-electroporation supplement to the cell suspension, wherein the post-electroporation supplement comprises a lipid enriched, human serum.

2. The method of claim 1, wherein viability of the cell is at least 30% greater than a cell that has not received the post-electroporation supplement.

3. The method of claim 1 or claim 2, wherein a viable number of cells is at least 1.5-fold greater than a cell that has not received the post-electroporation supplement.

4. The method of any one of claims 1 to 3, wherein the cell is a T-cell.

5. The method of any one of claims 1 to 3, wherein the cell is a hematopoietic stem cell.

6. The method of any one of claims 1 to 5, wherein the post-electroporation supplement comprises lipid enriched, human AB serum.

7. The method of any one of claims 1 to 6, wherein the post-electroporation supplement is added at about 2% to about 10% by volume of the cell suspension.

8. A method of transfecting a genetic construct into a cell, comprising:
a. suspending the cell in an electroporation buffer to create a cell suspension;
b. introducing the genetic construct into the cell suspension;
c. subjecting the cell to one or more electroporation pulses; and
d. adding a post-electroporation supplement to the cell suspension, wherein the post-electroporation supplement comprises a lipid enriched, human serum.

9. The method of claim 8, wherein viability of the cell is at least 30% greater than a cell that has not received the post-electroporation supplement.

10. The method of claim 8 or claim 9, wherein a viable number of cells is at least 1.5-fold greater than a cell that has not received the post-electroporation supplement.

11. The method of any one of claims 8 to 10, wherein a transfection efficiency of the cell is at least 15% greater than a cell that has not received the post-electroporation supplement.

12. The method of any one of claims 8 to 11, wherein the cell is a T-cell.

13. The method of any one of claims 8 to 11, wherein the cell is a hematopoietic stem cell.

14. The method of any one of claims 8 to 13, wherein the post-electroporation supplement comprises a lipid enriched, human AB serum.

15. The method of any one of claims 8 to 14, wherein the genetic construct is RNA or mRNA.

16. The method of claim 15, wherein the RNA is antisense RNA, siRNA or microRNA.

17. The method of any one of claims 8 to 14, wherein the genetic construct is DNA.

18. The method of any one of claims 8 to 14, wherein the genetic construct is a viral vector or a viral plasmid.

19. The method of any one of claims 8 to 14, wherein the genetic construct comprises a transposon.

20. The method of any one of claims 8 to 14, wherein the genetic construct comprises an endonuclease.

21. The method of any one of claims 8 to 20, wherein the post-electroporation supplement is added at about 2% to about 10% by volume of the cell suspension.

22. A composition comprising a lipid enriched, human AB serum, wherein the composition increases cell viability of an electroporated cell population.

23. The composition of claim 22, prepared by heating human AB serum to a temperature of about 60° C. to about 80° C. for about 20-45 minutes.

24. The composition of claim 22, prepared by passing human AB serum through a silica column and recovering the lipid enriched, human AB serum.
